(19) United States
(12) Patent Application Publication
(10) Pub. No.: US 2003/0199542 A1
(43) Pub. Date: Oct. 23, 2003

(54) Farnesyltransferase inhibitors

(76) Inventors: Keith W. Woods, Libertyville, IL (US); Qun Li, Libertyville, IL (US); Hing L. Sham, Vernon Hills, IL (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/125,015

(22) Filed: Apr. 18, 2002

(51) Int. Cl. A61K 31/4745; C07D 471/02

(52) U.S. Cl. 514/303; 546/119

(57) ABSTRACT

Compounds having the formula

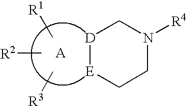

are farnesyltransferase inhibitors. Also disclosed are methods for making the compounds, pharmaceutical compositions containing the compounds, and methods of treatment using the compounds.

### TECHNICAL FIELD

The present invention provides substituted piperidines which inhibit farnesyltransferase, methods for making the compounds, pharmaceutical compositions containing the compounds, and methods of treatment using the compounds.

### BACKGROUND OF THE INVENTION

Ras oncogenes are the most frequently identified activated oncogenes in human tumors, and transformed protein Ras is involved in the proliferation of cancer cells. The Ras must be farnesylated by farnesyl pyrophosphate before this proliferation can occur, and farnesylation of Ras by farnesyl pyrophosphate is effected by protein farnesyltransferase. Inhibition of protein farnesyltransferase, and thereby farnesylation of the Ras protein, blocks the ability of transformed cells to proliferate.

Activation of Ras and related proteins which are farnesylated also partially mediates smooth muscle cell proliferation. Inhibition of protein isoprenyl transferases, and thereby farnesylation of the Ras protein, also aids in the prevention of intimal hyperplasia associated with restenosis and atherosclerosis, a condition which compromises the success of angioplasty and surgical bypass for obstructive vascular lesions.

Because of its pivitol role in tumor formation and metastasis, there has been continued interest in finding compounds that inhibit farnesyltransferase.

### SUMMARY OF THE INVENTION

In its principle embodiment, the present invention provides a compound of formula (I)

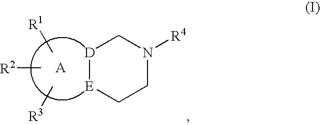

or a therapeutically acceptable salt thereof, wherein

A is a 5- or 6-membered aromatic or non-aromatic ring wherein from 0-3 carbon atoms are replaced by nitrogen;

D and E are independently selected from the group consisting of C and N;

with the proviso that when one of D and E is N, the other is C;

R¹ is selected from the group consisting of aryl and heteroaryl;

R² and R³ are selected from the group consisting of hydrogen, alkenyl, alkoxy, alkyl, amido, amino, cyano, halo, haloalkoxy, haloalkyl, nitro, and oxo; and

R⁴ is selected from the group consisting of arylalkyl and heteroarylalkyl.

In another embodiment the present invention discloses a pharmaceutical composition comprising a compound of formula (I) or a therapeutically acceptable salt thereof, in combination with a therapeutically acceptable carrier.

In another embodiment the present invention discloses a method for inhibiting farnesyltransferase in a patient in recognized need of such treatment comprising administering to the patient a therapeutically acceptable amount of a compound of formula (I), or a therapeutically acceptable salt thereof.

In another embodiment the present invention discloses a method for treating cancer in a patient in recognized need of such treatment comprising administering to the patient a therapeutically acceptable amount of a compound of formula (I), or a therapeutically acceptable salt thereof.

### DETAILED DESCRIPTION OF THE INVENTION

As used in the present specification the following terms have the meanings indicated:

The term “alkenyl,” as used herein, refers to a straight or branched chain group of one to twelve carbon atoms containing at least one carbon-carbon double bond.

The term “alkoxy,” as used herein, refers to an alkyl group attached to the parent molecular moiety through an oxygen atom.

The term “alkyl,” as used herein, refers to a straight or branched chain saturated hydrocarbon of one to twelve carbon atoms.

The term “amido,” as used herein, refers to an amino group attached to the parent molecular moiety through a carbonyl group.

The term “amino,” as used herein, refers to —NR^{a}R^{b}, wherein R^{a} and R^{b} are independently selected from the group consisting of hydrogen, alkyl, alkylcarbonyl, cycloalkyl, (cycloalkyl)alkyl, and unsubstituted phenyl.

The term “aryl,” as used herein, refers to a phenyl group, or a bicyclic or tricyclic fused ring system wherein one or more of the fused rings is a phenyl group. Bicyclic fused ring systems are exemplified by a phenyl group fused to a cycloalkenyl group, as defined herein, a cycloalkyl group, as defined herein, a heterocycle group, as defined herein, or another phenyl group. Tricyclic fused ring systems are exemplified by a bicyclic fused ring system fused to a cycloalkenyl group, as defined herein, cycloalkyl group, as defined herein, a heterocycle group, as defined herein, or another phenyl group. Representative examples of aryl include, but are not limited to, anthracenyl, azulenyl, benzodioxolyl,fluorenyl, indanyl, indenyl, naphthyl, phenyl, and tetrahydronaphthyl. The aryl groups of the present invention can be optionally substituted with one, two, three, four, or five substituents independently selected from the group consisting of alkenyl, alkoxy, alkyl, amino, cyano, halo, haloalkoxy, haloalkyl, nitro, and oxo.

The term “arylalkyl,” as used herein, refers to an aryl group attached to the parent molecular moiety through an alkyl group.

The term “carbonyl,” as used herein, refers to —C(O)—.

The term “cyano,” as used herein, refers to —CN.

The term “cycloalkenyl,” as used herein, refers to a non-aromatic cyclic or bicyclic ring system having three to ten carbon atoms and one to three rings, wherein each five-membered ring has one double bond, each six-membered ring has one or two double bonds, each seven- and eight-membered ring has one to three double bonds, and each nine-to ten-membered ring has one to four double bonds. Examples of cycloalkenyl groups include, but are not limited to, cyclohexenyl, octahydronaphthalenyl, and norbornylenyl.

The term “cycloalkyl,” as used herein, refers to a saturated monocyclic, bicyclic, or tricyclic hydrocarbon ring system having three to twelve carbon atoms. Examples of cycloalkyl groups include, but are not limited to, cyclopropyl, cyclopentyl, bicyclo[3.1.1]heptyl, and adamantyl.

The terms “halo,” and “halogen,” as used herein, refer to F, Cl, Br, or I.

The term “haloalkoxy,” as used herein, refers to a haloalkyl group attached to the parent molecular moiety through an oxygen atom.

The term “haloalkyl,” as used herein, refers to an alkyl group substituted by one, two, three, or four halogen atoms.

The term “heteroaryl,” as used herein, refers to an aromatic five- or six-membered ring where at least one atom is selected from the group consisting of N, O, and S, and the remaining atoms are carbon. The five-membered rings have two double bonds, and the six-membered rings have three double bonds. The heteroaryl groups are connected to the parent molecular group through a substitutable carbon or nitrogen atom in the ring. The term “heteroaryl” also includes systems where a heteroaryl ring is fused to an aryl group, as defined herein, a heterocycle group, as defined herein, or an additional heteroaryl group. Heteroaryls are exemplified by benzothienyl, benzoxadiazolyl, cinnolinyl, furanyl, imidazolyl, indazolyl, indolyl, isoxazolyl, isoquinolinyl, isothiazolyl, naphthyridinyl, oxadiazolyl, oxadiazolyl, oxazolyl, thiazolyl, thienopyridinyl, thienyl, triazolyl, thiadiazolyl, pyridinyl, pyridazinyl, pyrimidinyl, pyrazinyl, pyrazolyl, pyrrolyl, quinolinyl, triazinyl, and the like. The heteroaryl groups of the present invention can be optionally substituted with one, two, three, four, or five substituents independently selected from the group consisting of alkenyl, alkoxy, alkyl, amino, arylalkyl, cyano, halo, haloalkoxy, haloalkyl, nitro, and oxo.

The term “heteroarylalkyl,” as used herein, refers to a heteroaryl group attached to the parent molecular moiety through an alkyl group. The alkyl part of the heteroarylalkyl group can be optionally substituted with one or two aryl groups.

The term “heterocycle,” as used herein, refers to cyclic, non-aromatic, four-, five-, six-, or seven-membered rings containing at least one atom selected from the group consisting of oxygen, nitrogen, and sulfur. The four-membered rings have zero double bonds, the five-membered rings have zero or one double bonds, and the six- and seven-membered rings have zero, one, or two double bonds. Heterocycle groups of the invention are exemplified by dihydropyridinyl, 1,3-dioxanyl, 1,4-dioxanyl, 1,3-dioxolanyl, morpholinyl, piperazinyl, pyrrolidinyl, tetrahydropyridinyl, piperidinyl, thiomorpholinyl, and the like.

The term “nitro,” as used herein, refers to —NO₂.

The term “oxo,” as used herein, refers to ═O.

The compounds of the present invention can exist as therapeutically acceptable salts. The term “therapeutically acceptable salt,” as used herein, represents salts or zwitterionic forms of the compounds of the present invention which are water or oil-soluble or dispersible, which are suitable for treatment of diseases without undue toxicity, irritation, and allergic response; which are commensurate with a reasonable benefit/risk ratio, and which are effective for their intended use. The salts can be prepared during the final isolation and purification of the compounds or separately by reacting an amino group with a suitable acid. Representative acid addition salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, fumarate, hydrochloride, hydrobromide, hydroiodide, 2-hydroxyethansulfonate, lactate, maleate, mesitylenesulfonate, methanesulfonate, naphthylenesulfonate, nicotinate, 2-naphthalenesulfonate, oxalate, pamoate, pectinate, persulfate, 3-phenylproprionate, picrate, pivalate, propionate, succinate, tartrate, trichloroacetate,trifluoroacetate, phosphate, glutamate, bicarbonate, paratoluenesulfonate, and undecanoate. Also, amino groups in the compounds of the present invention can be quatemized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobrornic, sulfuric, and phosphoric, and organic acids such as oxalic, maleic, succinic, and citric.

The present compounds can also exist as therapeutically acceptable prodrugs. The term “therapeutically acceptable prodrug,” refers to those prodrugs or zwitterions which are suitable for use in contact with the tissues of patients without undue toxicity, irritation, and allergic response, are commensurate with a reasonable benefit/risk ratio, and are effective for their intended use. The term “prodrug,” refers to compounds which are rapidly transformed in vivo to parent compounds of formula (I) for example, by hydrolysis in blood.

Asymmetric centers exist in the compounds of the present invention. These centers are designated by the symbols “R” or “S,” depending on the configuration of substituents around the chiral carbon atom. It should be understood that the invention encompasses all stereochemical isomeric forms, or mixtures thereof, which possess the ability to inhibit farnesyltransferase. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, or direct separation of enantiomers on chiral chromatographic columns. Starting compounds of particular stereochemistry are either commercially available or can be made and resolved by techniques known in the art.

In accordance with methods of treatment and pharmaceutical compositions of the invention, the compounds can be administered alone or in combination with other farnesyltransferase inhibitors. When using the compounds, the specific therapeutically effective dose level for any particular patient will depend upon factors such as the disorder being treated and the severity of the disorder; the activity of the particular compound used; the specific composition employed; the age, body weight, general health, sex, and diet of the patient; the time of administration; the route of administration; the rate of excretion of the compound employed; the duration of treatment; and drugs used in combination with or coincidently with the compound used. The compounds can be administered orally, parenterally, osmotically (nasal sprays), rectally, vaginally, or topically in unit dosage formulations containing carriers, adjuvants, diluents, vehicles, or combinations thereof. The term “parenteral” includes infusion as well as subcutaneous, intravenous, intramuscular, and intrasternal injection.

Parenterally administered aqueous or oleaginous suspensions of the compounds can be formulated with dispersing, wetting, or suspending agents. The injectable preparation can also be an injectable solution or suspension in a diluent or solvent. Among the acceptable diluents or solvents employed are water, saline, Ringer's solution, buffers, monoglycerides, diglycerides, fatty acids such as oleic acid, and fixed oils such as monoglycerides or diglycerides.

The inhibitory effect of parenterally administered compounds can be prolonged by slowing their absorption. One way to slow the absorption of a particular compound is administering injectable depot forms comprising suspensions of crystalline, amorphous, or otherwise water-insoluble forms of the compound. The rate of absorption of the compound is dependent on its rate of dissolution which is, in turn, dependent on its physical state. Another way to slow absorption of a particular compound is administering injectable depot forms comprising the compound as an oleaginous solution or suspension. Yet another way to slow absorption of a particular compound is administering injectable depot forms comprising microcapsule matrices of the compound trapped within liposomes, microemulsions, or biodegradable polymers such as polylactide-polyglycolide, polyorthoesters or polyanhydrides. Depending on the ratio of drug to polymer and the composition of the polymer, the rate of drug release can be controlled.

Transdermal patches can also provide controlled delivery of the compounds. The rate of absorption can be slowed by using rate controlling membranes or by trapping the compound within a polymer matrix or gel. Conversely, absorption enhancers can be used to increase absorption.

Solid dosage forms for oral administration include capsules, tablets, pills, powders, and granules. In these solid dosage forms, the active compound can optionally comprise diluents such as sucrose, lactose, starch, talc, silicic acid, aluminum hydroxide, calcium silicates, polyamide powder, tableting lubricants, and tableting aids such as magnesium stearate or microcrystalline cellulose. Capsules, tablets and pills can also comprise buffering agents, and tablets and pills can be prepared with enteric coatings or other release-controlling coatings. Powders and sprays can also contain excipients such as talc, silicic acid, aluminum hydroxide, calcium silicate, polyamide powder, or mixtures thereof. Sprays can additionally contain customary propellants such as chlorofluorohydrocarbons or substitutes therefore.

Liquid dosage forms for oral administration include emulsions, microemulsions, solutions, suspensions, syrups, and elixirs comprising inert diluents such as water. These compositions can also comprise adjuvants such as wetting, emulsifying, suspending, sweetening, flavoring, and perfuming agents.

Topical dosage forms include ointments, pastes, creams, lotions, gels, powders, solutions, sprays, inhalants, and transdermal patches. The compound is mixed under sterile conditions with a carrier and any needed preservatives or buffers. These dosage forms can also include excipients such as animal and vegetable fats, oils, waxes, paraffins, starch, tragacanth, cellulose derivatives, polyethylene glycols, silicones, bentonites, silicic acid, talc and zinc oxide, or mixtures thereof. Suppositories for rectal or vaginal administration can be prepared by mixing the compounds with a suitable non-irritating excipient such as cocoa butter or polyethylene glycol, each of which is solid at ordinary temperature but fluid in the rectum or vagina. Ophthalmic formulations comprising eye drops, eye ointments, powders, and solutions are also contemplated as being within the scope of this invention.

The total daily dose of the compounds administered to a host in single or divided doses can be in amounts from about 0.1 to about 200 mg/kg body weight or preferably from about 0.25 to about 100 mg/kg body weight. Single dose compositions can contain these amounts or submultiples thereof to make up the daily dose.

Determination of Biological Activity

The ability of the compounds of the present invention to inhibit farnesyltransferase can be measured according to the method described in J. Biol. Chem., 266: 14603 (1991) or J. Biol. Chem., 270:660-664 (1995). Procedures for determination of the inhibition of farnesylation of the oncogene protein Ras are described in J. Biol. Chem., 266:15575-15578 (1991) and U.S. Pat. No. 5,245,061. Inhibition of rat brain farnesyltransferase can also be measured in vitro using an Amersham Life Science commercial scintillation proximity assay kit and substituting a biotin-K Ras B fragment (0.1M final concentration) for the biotin-lamin substrate provided by Amersham. The enzyme can be purified according to Cell, 62: 81-88 (1990), utilizing steps one, two, and three. The specific activity of the enzyme is approximately 10 nmol substrate farnesylated/mg of enzyme/hour. The percent inhibition of the farnesylation caused by the compounds of the present invention (at 10⁻⁷M) compared to an uninhibited control sample can be evaluated in the same Amersham test system.

Briefly, ³H-Farnesyldiphosphate (final concentration 0.6M), H-Ras (final concentration 5.0 μM), and the test compound (various final concentrations from a stock solution in 50% DMSO/water; final concentration DMSO<2%) were mixed in a buffer comprising 50 mM HEPES (pH 7.5), 30 mM MgCl₂, 20 mM KCl, 10 μM ZnCl₂, 5 mM DTT, and 0.01% Triton X-100) to give a final volume of 50 μL. The mixture was brought to 37° C., treated with enzyme, incubated for 30 minutes, treated with 1M HCl/ethanol (1 mL) to stop the reaction, stirred for 15 minutes at room temperature, diluted with ethanol (2 mL), filtered through a 2.5 cm glass microfiber filter (Whatman) with ethanol rinses (4×2 mL). The glass filter was transferred to a scintillation vial and treated with scintillation fluid (5 mL). The radioisotope retained on the glass fiber filter was counted and reflected the activity of the enzyme. The percent inhibition of farnesyltransferase was determined for the compounds of the present invention at concentrations of 10⁻⁷M. The compounds inhibited farnesyltransferase at percentages of between about 80% and about 97%.

Therefore, the compounds of the present invention, including but not limited to those specified in the examples, are useful for the treatment of diseases caused or exascerbated by farnesyltransferase. As farnesyltransferase inhibitors, these compounds are useful in the treatment of both primary and metastatic solid tumors and carcinomas of the breast; colon; rectum; lung; oropharynx; hypopharynx; esophagus; stomach; pancreas; liver; gallbladder; bile ducts; small intestine; urinary tract (kidney, bladder, and urothelium); female genital tract (cervix, uterus, and ovaries); male genital tract (prostate, seminal vesicles, and testes); endocrine glands (thyroid, adrenal, and pituitary); skin (hemangiomas, melanomas, and sarcomas); tumors of the brain, nerves, and eyes; meninges (astrocytomas, gliomas, glioblastomas, retinoblastomas, neuromas, neuroblastomas, and meningiomas); solid tumors arising from hematopoietic malignancies (leukemias and chloromas); plasmacytomas; plaques; tumors of mycosis fungoides; cutaneous T-cell lymphoma/leukemia; lymphomas including Hodgkin's and non-Hodgkin's lymphomas; prophylaxis of autoimmune diseases (rheumatoid, immune and degenerative arthritis); ocular diseases (diabetic retinopathy, retinopathy of prematurity, corneal graft rejection, retrolental fibroplasia, neovascular glaucoma, rubeosis, retinal neovascularization due to macular degeneration, and hypoxia); skin diseases (psoriasis, hemagiomas and capillary proliferation within atherosclerotic plaques).

Synthetic Methods

Abbreviations which have been used in the descriptions of the scheme and the examples that follow are: PPh₃ for triphenylphosphine; DMF for N,N-dimethylformamide; DMA for N,N-dimethylacetamide; LDA for lithium diisopropylarmide; NBS for N-bromosuccinimide; THF for tetrahydrofuran; and DMSO for dimethylsulfoxide.

The compounds and processes of the present invention will be better understood in connection with the following synthetic schemes which illustrate the methods by which the compounds of the invention may be prepared. The compounds of the present invention can be prepared by a variety of synthetic routes. Representative procedures are shown in Schemes 1-13. Starting materials can be obtained from commercial sources or prepared by well-established literature methods known to those of ordinary skill in the art. The groups D, E, R¹, R², R³, and R⁴ are as defined above unless otherwise noted below. It will be apparent that protection and deprotection steps, as well as the order of the steps themselves, can be carried out in varying order to successfully complete the syntheses of compounds of the present invention.

The reactions described in the schemes are carried out in reaction inert solvents. The expression “reaction inert solvent” refers to a solvent or mixture of solvents which does not interact with starting materials, reagents, intermediates, or products in a manner which adversely affects the yield of the desired product.

This invention is intended to encompass compounds having formula (I) when prepared by synthetic processes or by metabolic processes. Preparation of the compounds of the invention by metabolic processes include those occurring in the human or animal body (in vivo) or processes occurring in vitro.

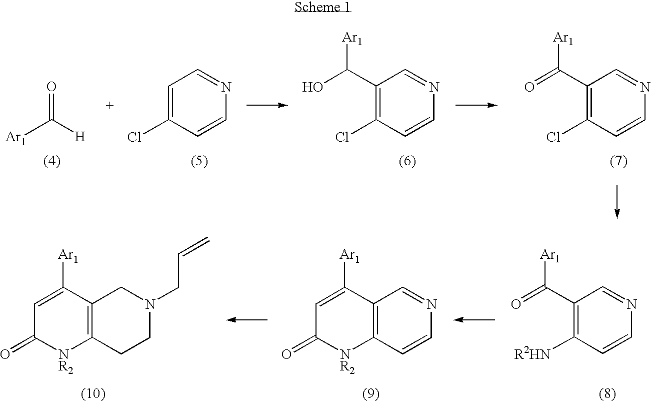

Scheme 1 shows the synthesis of compounds of formula (10). Compounds of formula (4) can be reacted with compounds of formula (5) in the presence of a base to provide compounds of formula (6). Examples of bases include lithium diisopropylamine, lithium hexamethyldisilazide, and potassium hexamethyldisilazide. Compounds of formula (6) can be converted to compounds of formula (7) under oxidative conditions. Representative oxidizing agents include MnO₂ and NaIO₄ with catalytic RuO₂. Conversion of compounds of formula (7) to compounds of formula (8) (R₂ is alkyl or alkenyl) can be accomplished by treatment with a primary amine under increased pressures and/or temperatures. The amine can then be cyclized by treatment with tert-butyl acetate and a base such as lithium diisopropylamine or lithium hexamethyldisilazide to provide a compound of formula (9). This compound can be reduced to provide a compound of formula (10) by treatment with allyl bromide followed by treatment with sodium borohydride and sodium cyanoborohydride.

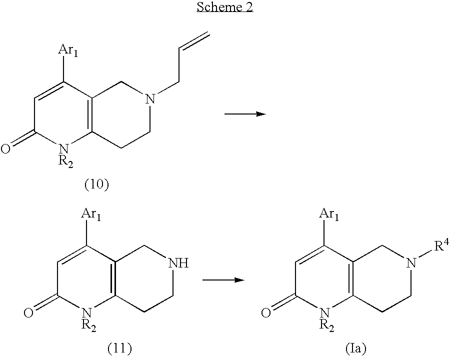

As shown in Scheme 2, compounds of formula (10) can be deprotected with a rhodium catalyst such as (PPh₃)₃RhCl to provide compounds of formula (11). Reaction of compounds of formula (11) with an arylalkyl halide or a heteroarylalkyl halide in the presence of a base such as diisopropylamine or triethylamine provides compounds of formula (Ia).

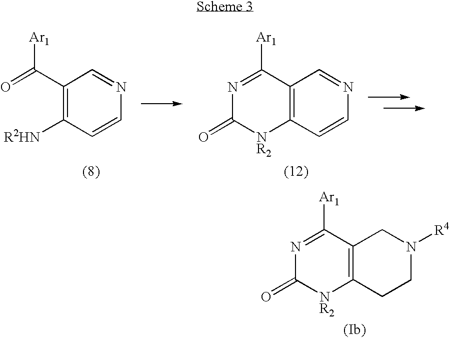

Scheme 3 shows the synthesis of compounds of formula (Ib). Compounds of formula (8) (prepared by the procedure described in Scheme 1) can be treated with chlorosulfonyl isocyanate to provide compounds of formula (12). These compounds can then be coverted to compounds of formula (Ib) using the conditions described in Schemes 1 and 2.

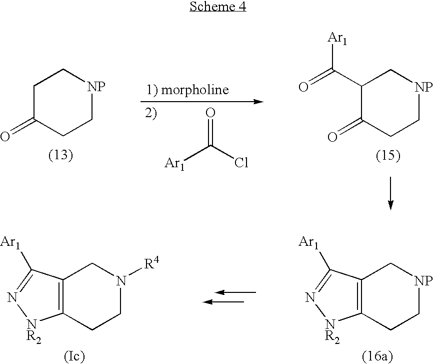

Compounds of formula (Ic) can be synthesized following the sequence described in Scheme 4. Compounds of formula (13) (P is a protecting group such as acetyl) can be treated with morpholine followed by compounds of formula (14) in the presence of a base such as triethylamine to provide compounds of formula (15). Cyclization of compounds of formula (15) can be accomplished by treatment with hydrazine to provide compounds of formula (16a) (R² is hydrogen). Compounds of formula (16a) where R² is hydrogen can be converted to compounds of formula (16a) where R² is alkyl by treatment with a base such as sodium hydride and an alkylating agent such as an alkyl halide. Removal of the protecting group (P) in compounds of formula (16a) using conditions known to those of ordinary skill in the art provides the free amine which can be converted to compounds of formula (Ic) following the procedures described in Scheme 2.

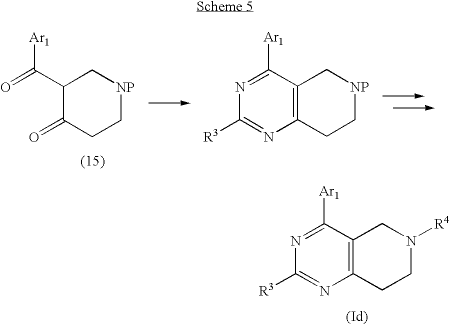

As shown in Scheme 5, compounds of formula (15) (prepared according to the procedures described in Scheme 4) can be converted to compounds of formula (16b) by treatment with pyrrolidine followed by treatment with an appropriately substituted imidamide (R³C(NH)NH₂, where R³ is hydrogen, alkyl, or alkenyl). Upon removal of the protecting group (P), these compounds can be converted to compounds of formula (Id) by the methods described in Scheme 2.

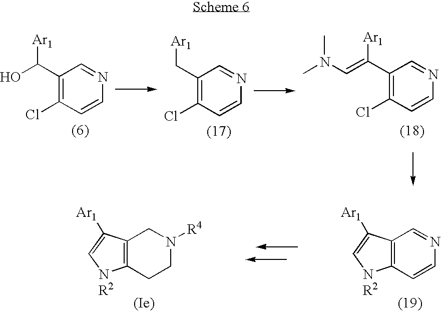

The synthesis of compounds of formula (Ie) is shown in Scheme 6. Compounds of formula (6) (prepared according to the procedure described in Scheme 1) can be treated with 1,1′-thiocarbonyldiimidazole then treated with tributyltin hydride to provide compounds of formula (17). Reaction of these compounds with dimethylformamide dimethylacetal provides compounds of formula (18), which can be cyclized to provide compounds of formula (19) (R² is alkyl or alkenyl) upon treatment with an appropriately substituted amine. Compounds of formula (19) can be converted to compounds of formula (Ie) using the methods described in Scheme 2.

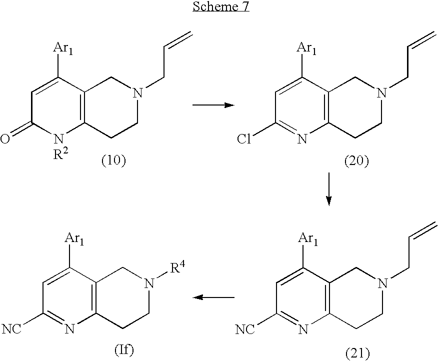

As shown in Scheme 7, compounds of formula (10) (prepared according to the methods described in Scheme 1) can be treated with POCl₃ to provide compounds of formula (20). Treatment of compounds of formula (20) with a cyanide source such as sodium cyanide or potassium cyanide provides compounds of formula (21), which can then be converted to compounds of formula (If) using the conditions described in Scheme 2.

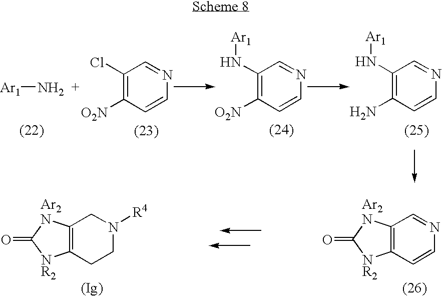

Scheme 8 shows the synthesis of compounds of formula (Ig). Compounds of formula (22) (Ar₁ is aryl or heteroaryl) can be reacted with compounds of formula (23) to provide compunds of (24). The nitro group can then be reduced to an amino group, providing compounds of formula (25), using reducing conditions known to those of ordinary skill in the art (e.g. tin (II) chloride). Compounds of formula (25) can be cyclized to provide compounds of formula (26) (R² is hydrogen) by reaction with 1,1′-carbonyldiimidazole or an equivalent acylating agent. Compounds of formula (26) where R² is hydrogen can be converted to compounds of formula (26) where R² is alkyl or alkenyl by treatment with an alkylating agent such as an alkyl halide or an alkyl tosylate. Compounds of formula (26) can be converted to compounds of formula (Ig) using the procedures described in Schemes 1 and 2.

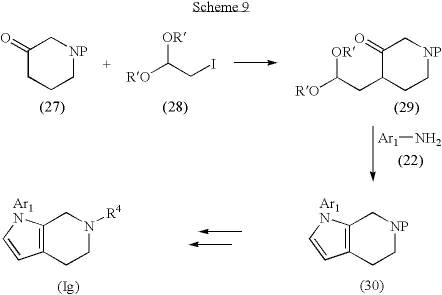

As shown in Scheme 9, compounds of formula (27) (P is a protecting group such as an acetyl group) can be reacted with a base such as LDA then treated with compounds of formula (28) (R′ is alkyl) to provide compounds of formula (29). Cyclization of compounds of formula (29) can be accomplished by treatment with treatment with compounds of formula (22) (Ar₁ is aryl or heteroaryl) in the presence of an acid such as p-toluenesulfonic acid to provide compounds of formula (30). Upon removal of the protecting group (P), these compounds can be converted to compounds of formula (Ig) using the conditions described in Scheme 2.

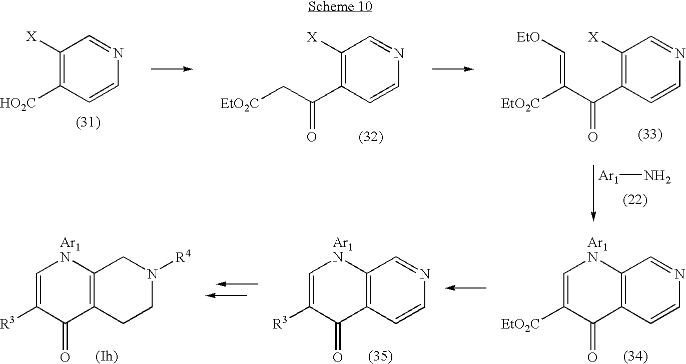

Scheme 10 shows the synthesis of compounds of formula (Ih). Compounds of formula (31) (X is halo) can be converted to compounds of formula (32) by treatment with mono-ethyl malonate. Generation of the enolate followed by condensation with triethyl orthoformate provides a compound of formula (33) which can be cyclized to a compound of formula (34) by treatment with a compound of formula (22) (Ar₁ is aryl or heteroaryl) in the presence of a base such as sodium hydride. Compounds of formula (34) can be converted to compounds of formula (35) where R³ is cyano by hydrolysis of the ester under basic conditions, followed by treatment with 1,1′-carbonyldiimidazole and ammonia and treatment with POCl₃. Alternatively, compounds of formula (34) can be converted to compounds of formula (35) where R³ is amido by hydrolysis under basic conditions followed by treatment with an appropriately substituted amine. Compounds of formula (35) can be converted to compounds of formula (Ih) by the methods described in Schemes 1 and 2.

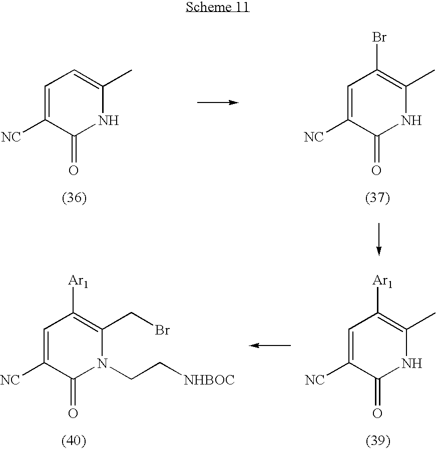

As shown in Scheme 11, compounds of formula (36) can be converted to compounds of formula (37) by bromination with NBS. These compounds can be coupled with an appropriately substituted aryl or heteroaryl boronic acid or ester in the presence of a palladium catalyst such as tetrakistriphenylphosphine palladium(0) and a base such as cesium carbonate to provide compounds of formula (39). N-Alkylation with N-tert-butoxycarbonyl-2-iodoethylamine in the presence of a base such as cesium carbonate followed by allylic bromination of the methyl group with NBS provides compounds of formula (40).

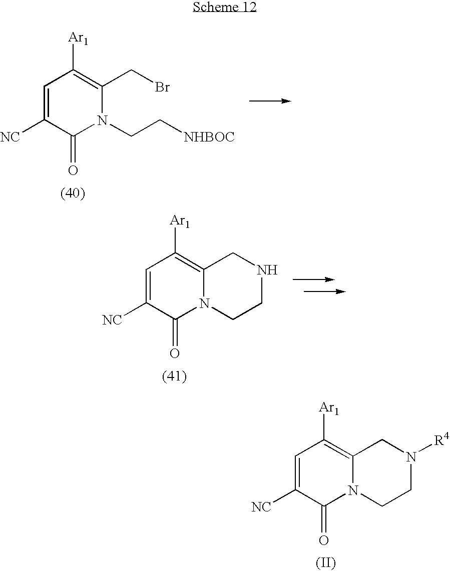

Scheme 12 shows the synthesis of compounds of formula (Ii). Compounds of formula (40) can be cyclized to provide compounds of formula (41) by removal of the nitrogen protecting group under acidic conditions (such as p-toluenesulfonic acid) followed by treatment with a base such as potassium carbonate. Compounds of formula (41) can be converted to compounds of formula (Ii) using the conditions described in Scheme 2.

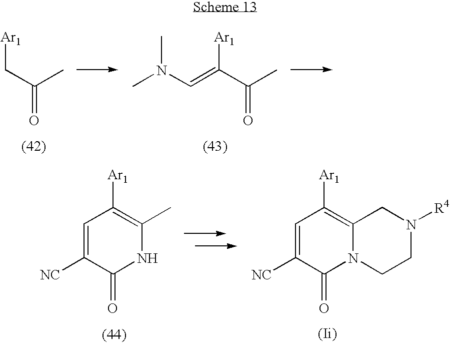

An alternative synthesis of compounds of formula (Ii) is shown in Scheme 13. Compounds of formula (42) can be treated with dimethylformamide dimethylacetal to provide compounds of formula (43). These compounds can be cyclized to provide compounds of formula (44) by treatment with 2-cyanoacetamide and a base such as sodium methoxide or sodium hydride. Compounds of formula (44) can be converted to compounds of formula (Ii) using the methods described in Schemes 11, 12, and 2.

The present invention will now be described in connection with certain preferred embodiments which are not intended to limit its scope. On the contrary, the present invention covers all alternatives, modifications, and equivalents as can be included within the scope of the claims. Thus, the following examples, which include preferred embodiments, will illustrate the preferred practice of the present invention, it being understood that the examples are for the purposes of illustration of certain preferred embodiments and are presented to provide what is believed to be the most useful and readily understood description of its procedures and conceptual aspects.

Compounds of the invention were named by ACD/ChemSketch version 5.0 (developed by Advanced Chemistry Development, Inc., Toronto, ON, Canada) or were given names which appeared to be consistent with ACD nomenclature.

### EXAMPLE 1

4-[[4-(3-chlorophenyl)-1-methyl-2-oxo-1,5,7,8-tetrahydro-1,6-naphthyridin-6(2H)-yl](1-methyl-1H-imidazol-5-yl)methyl]benzonitrile

### EXAMPLE 1A

(3-chlorophenyl)(4-chloropyridin-3-yl)methanol

A solution of 0.6M LDA in THF (110 mL, 66 mmol) at −78° C. was treated with a solution of 4-chloropyridine (6.3 g, 55.5 mmol) in THF (50 mL), stirred for 1.5 hours, treated with a solution of 3-chlorobenzaldehyde (8.7 g, 61.8 mmol) in THF (30 mL), stirred for 2 hours, warmed to room temperature, and stirred for 18 hours. The reaction was adjusted to pH<7 with saturated NH₄Cl (aq) and extracted three times with ethyl acetate. The combined extracts were washed with brine, dried (Na₂SO₄), filtered, and concentrated. The concentrate was recrystallized from ethyl acetate/hexanes, filtered, and washed with diethyl ether to provide 10.8 g (77%) of the desired product.

### EXAMPLE 1B

(3-chlorophenyl)(4-chloropyridin-3-yl)methanone

A solution of Example 1A (7.5 g, 29.5 mmol) in dioxane (50 mL) was treated with MnO₂ (13 g, 149.5 mmol), heated to reflux for 1.5 hours, and filtered through diatomaceous earth (Celite®). The pad was washed with ethyl acetate and the combined filtrates were concentrated to provide 7.1 g (96%) of the desired product.

### EXAMPLE 1C

(3-chlorophenyl)[4-(methylamino)pyridin-3-yl]methanone

A mixture of Example 1B (7.4 g, 29.5 mmol) and 40 wt % CH₃NH₂ in water (50 mL) in a pressure tube was heated to 150° C. and cooled to room temperature. The mixture was diluted with water and extracted four times with dichloromethane. The combined extracts were washed with brine, dried (Na₂SO₄), filtered, and concentrated. The concentrate was dissolved in THF (80 mL) and H₂O (20 mL), treated with conc. HCl (0.5 mL), heated to reflux for 3 hours, neutralized with saturated NaHCO₃ (aq), and extracted three times with ethyl acetate. The combined extracts were washed with water and brine, dried (MgSO₄), filtered, and concentrated. The concentrate was recrystallized from ethyl acetate/hexanes to provide 4 g (55%) of the desired product.

### EXAMPLE 1D

4-(3-chlorophenyl)-1-methyl-1,6-naphthyridin-2(1H)-one

A solution of 0.6M IDA in THF (40 mL, 24 mmol) at −78° C. was treated with tert-butyl acetate (3.8 g, 33 mmol), stirred for 30 minutes, treated with a solution of Example 1C (2.0 g, 8.1 mmol) in THF (25 mL), stirred for 3 hours, warmed to room temperature, and stirred for 18 hours. The mixture was diluted with water and extracted three times with ethyl acetate. The combined extracts were washed with brine, dried (MgSO₄), filtered, and concentrated. The concentrate was purified by flash column chromatography on silica gel with 3% methanolldichloromethane to provide 1.7 g, (78%) of the desired product.

### EXAMPLE 1E

6-allyl-4-(3-chlorophenyl)-1-methyl-2-oxo-1,2-dihydro-1,6-naphthyridin-6-ium bromide

A solution of Example 1D (270 mg, 1.0 mmol) in allyl bromide (5 mL) was heated to reflux for 3 hours, cooled to room temperature, and diluted with diethyl ether. The precipitate was collected by filtration, rinsed with diethyl ether, and dried under vacuum to provide 380 mg (97%) of the desired product.

### EXAMPLE 1F

6-allyl-4-(3-chlorophenyl)-1-methyl-5,6,7,8-tetrahydro-1,6-naphthyridin-2(1H)-one

A solution of Example 1E (378 mg, 0.96 mmol) in methanol (10 mL) and water (0.5 mL), was treated slowly with NaBH₄ (150 mg, 4.0 mmol), stirred for 15 minutes, treated with conc. HCl (15 drops), treated portionwise with NaCNBH₃ (300 mg, 4.77 mmol), stirred for 30 minutes, and concentrated. The concentrate was partitioned between saturated NaHCO₃ (aq) and ethyl acetate and the aqueous layer was extracted three times with ethyl acetate. The combined extracts were washed with brine, dried (Na₂SO₄), filtered, and concentrated. The concentrate was purified by flash column chromatography on silica gel with 5% methanol/dichloromethane to provide 260 mg (86%) of the desired product.

### EXAMPLE 1G

4-(3-chlorophenyl)-1-methyl-5,6,7,8-tetrahydro-1,6-naphthyridin-2(1H)-one

A mixture of Example 1F (245 mg, 0.78 mmol) and (PPh₃)₃RhCl (35 mg, 0.04 mmol) in 85:15 CH₃CN:H₂O (20 mL) was added to a two neck round-bottom flask fitted with a reflux condenser and a short-path distillation head. The mixture was heated to reflux for 4 hours, cooled to room temperature, stirred for 18 hours, and concentrated. The concentrate was purified by flash column chromatography on silica gel with 10% methanol/dichloromethane to provide 130 mg (61%) of the desired product.

### EXAMPLE 1H

4-[hydroxy(1-methyl-1H-imidazol-5-yl)methyl]benzonitrile

A solution of 1-methyl-5-(triethylsilyl)-1H-imidazole (3.35 g, 17.08 mmol) in THF (50 mL) at −78° C. was treated dropwise with 2.5M tert-butyllithium in pentane (22.4 mL, 17.1 mmol), stirred for 30 minutes, treated dropwise with a solution of 4-cyanobenzaldehyde (2.04 g, 15.56 mmol) in THF (10 mL), and stirred for 1 hour. The mixture was quenched with methanol (4 mL), treated with 1N HCl (40 mL), warmed to room temperature, adjusted to pH 12 with 30% NaOH, and extracted with ethyl acetate. The combined extracts were washed with brine, dried (MgSO₄), filtered, and concentrated. The concentrate was triturated with 4:1 hexanes/ethyl acetate to provide 2.95 g (89%) of the desired product.

### EXAMPLE 1I

4-[chloro(1-methyl-1H-imidazol-5-yl)methyl]benzonitrile

A solution of Example 1H (1.42 g, 6.66 mmol) in dichloromethane (40 mL) at 0° C. was treated with SOCl₂ (2.8 mL, 38.4 mmol), warmed to room temperature, stirred for 4 hours, and concentrated. The concentrate was azeotropically distilled with toluene to provide 2.0 g (quantitative) of the desired product.

### EXAMPLE 1J

4-[[4-(3-chlorophenyl)-1-methyl-2-oxo-1,5,7,8-tetrahydro-1,6-naphthyridin-6(2H-yl](1-methyl-1H-imidazol-5-yl)methyl]benzonitrile

A solution of Example 1G (125 mg, 0.45 mmol) and Example 1I (200 mg, 0.74 mmol) in CH₃CN (3 mL) was treated with diisopropylethylamine (185 mg, 1.43 mmol), heated overnight at 50-60° C., and concentrated. The concentrate was partitioned between water and ethyl acetate and the aqueous phase was extracted three times with ethyl acetate. The combined extracts were washed with brine, dried (MgSO₄), filtered, and concentrated. The concentrate was purified by flash column chromatography on silica gel with 5% methanol/dichloromethane, dissolved in dichloromethane, and treated with 1.0M HCl in diethyl ether (1 mL). The solution was stirred for 1 hour and concentrated to provide 51 mg (22%) of the desired product as the hydrochloride salt. MS (DCI) m/z 470 (M+H)⁺; ¹H NMR (300 MHz, DMSO-d₆) δ7.74 (d, J=8 Hz, 2H), 7.52 (d, J=8 Hz, 2H), 7.47 (s, 1H), 7.32-7.43 (m, 2H), 7.17-7.19 (m, 1H), 7.08-7.12 (m, 1H), 6.78 (s, 1H), 6.13 (s, 1H), 4.92 (s, 1H), 3.48 (s, 3H), 3.45 (s, 3H), 2.94-3.08 (m, 2H), 2.63-2.90 (m, 4H); Anal. Calcd for C₂₇H₂₄ClN₅O.1.5HCl: C, 61.81; H, 4.90; N, 13.35; Cl, 16.89. Found: C, 61.74; H, 4.96; N, 13.10; Cl, 16.33.

### EXAMPLE 2

4- [(5-{[4-(3-chlorophenyl)-1-methyl-2-oxo-1,5,7,8-tetrahydro-1,6-naphthyridin-6(2H)-yl]methyl}-1H-imidazol-1-yl)methyl]benzonitrile

The desired product was prepared as the free base by substituting 4-{[5-(chloromethyl)-1H-imidazol-1-yl]methyl}benzonitrile (prepared according to the procedure described in WO 00/01691) for Example 1I in Example 1J. Purification of the crude product by flash column chromatography on silica gel with 5% methanol/dichloromethane provided the desired product. MS (DCI) m/z 470 (M+H)⁺; ¹H NMR (300 MHz, DMSO-d₆) δ7.73 (d, J=2 Hz, 1H), 7.65 (d, J=9 Hz, 2H), 7.46-7.49 (m, 2H), 7.36-7.38 (m, 1H), 7.21-7.27 (m, 1H), 7.13 (d, J=9 Hz, 2H), 6.77 (d, J=2 Hz, 1H), 6.11 (s, 1H), 5.28 (s, 2H), 3.39 (s, 3H), 3.36 (s, 2H), 2.94 (s, 2H), 2.49-2.54 (m, 4H); Anal. Calcd for C₂₇H₂₄ClN₅O.0.25H₂O: C, 68.31; H, 5.20; N, 14.75. Found: C, 68.32; H, 5.13; N, 14.71.

### EXAMPLE 3

4-[[4-(3-methoxyphenyl)-1-methyl-2-oxo-1,5,7,8-tetrahydro-1,6-naphthyridin-6(2H)-yl]1-methyl-1H-imidazol-5-yl)methyl]benzonitrile

### EXAMPLE 3A

4-(3-methoxyphenyl)-1-methyl-5,6,7,8-tetrahydro-1,6-naphthyridin-2(1H)-one

The desired product was prepared by substituting 3-methoxybenzaldehyde for 3-chlorobenzaldehyde in Examples 1A-1G.

### EXAMPLE 3B

4-[[4-(3-methoxyphenyl)-1-methyl-2-oxo-1,5,7,8-tetrahydro-1,6-naphthyridin-6(2H)-yl](1-methyl-1H-imidazol-5-yl)methyl]benzonitrile

The desired product was prepared as the free base by substituting Example 3A for Example 1G in Example 1J. MS (DCI) m/z 466 (M+H)⁺; ¹H NMR (300 MHz, CDCl₃) δ7.60 (d, J=9 Hz, 2H), 7.44 (d, J=9 Hz, 2H), 7.35-7.37 (m, 1H), 7.18-7.23 (m, 1H), 6.97 (s, 1H), 6.83-6.88 (m, 2H), 6.58-6.67 (m, 1H), 6.40 (s, 1H), 4.70 (s, 1H), 3.79 (s, 3H), 3.57 (s, 3H), 3.44 (s, 3H), 3.05-3.19 (m, 2H), 2.71-2.90 (m, 4H); Anal. Calcd for C₂₈H₂₇N₅O₂.0.5H₂O: C, 70.87; H, 5.95; N, 14.76. Found: C, 71.14; H, 5.95; N, 14.57.

### EXAMPLE 4

4-[(5-{[4-(3-methoxyphenyl)-1-methyl-2-oxo-1,5,7,8-tetrahydro-1,6-naphthyridin-6(2H)-yl]methyl}-1H-imidazol-1-yl)methyl]benzonitrile

The desired product was prepared as the free base by substituting Example 3A and 4-{[5-(chloromethyl)-1H-imidazol-1-yl]methyl}benzonitrile (prepared according to the procedure described in WO 00/01691) for Example 1G and Example 1I, respectively, in Example 1J. MS (ESI) m/z 466 (M+H)⁺; ¹H NMR (CDCl₃, 300 MHz) δ7.55 (d, J=9 Hz, 2H), 7.53 (s, 1H), 7.30-7.37 (m, 1H), 7.03 (d, J=9 Hz, 2H), 6.92-6.97 (m, 2H), 6.74-6.80 (m, 2H), 6.42 (s, 1H), 5.23 (s, 2H), 3.87 (s, 3H), 3.52 (s, 3H), 3.26 (s, 2H), 3.08 (s, 2H), 2.64 (s, 4H).

### EXAMPLE 5

4-[[4-(1,3-benzodioxol-5-yl)-1-methyl-2-oxo-1,5,7,8-tetrahydro-1,6-naphthyridin-6(2H)-yl](1-methyl-1H-imidazol-5-yl)methyl]benzonitrile

### EXAMPLE 5A

4-(1,3-benzodioxol-5-yl)-1-methyl-5,6,7,8-tetrahydro-1,6-naphthyridin-2(1H)-one

The desired product was prepared by substituting 1,3-benzodioxole-5-carbaldehyde for 3-chlorobenzaldehyde in Examples 1A-1G.

### EXAMPLE 5B

4-[[4-(1,3-benzodioxol-5-yl)-1-methyl-2-oxo-1,5,7,8-tetrahydro-1,6-naphthyridin-6(2H)-yl](1-methyl-1H-imidazol-5-yl)methyl]benzonitrile

The desired product was prepared as the free base by substituting Example 5A for Example 1G in Example 1J. MS (DCI) m/z 480 (M+H)⁺; ¹H NMR (300 MHz, CDCl₃) δ7.61 (d, J=9 Hz, 2H), 7.54 (s, 1H), 7.44 (d, J=9 Hz, 2H), 7.03 (s, 1H), 6.72-6.76 (m, 1H), 6.51-6.57 (m, 2H), 6.38 (s, 1H), 5.98-6.01 (m, 2H), 4.71 (s, 1H), 3.55 (s, 3H), 3.51 (s, 3H), 3.06-3.20 (m, 2H), 2.72-2.80 (m, 4H); Anal. Calcd for C₂₈H₂₅N₅O₃.0.5H₂O: C, 68.84; H, 5.36; N, 14.39. Found: C, 69.11; H, 5.29; N, 13.99.

### EXAMPLE 6

4-[(5-{[4-(1,3-benzodioxol-5-yl)-1-methyl-2-oxo-1,5,7,8-tetrahydro-1,6-naphthyridin-6(2H)-yl]methyl}-1H-imidazol-1-yl)methyl]benzonitrile

The desired product was prepared as the free base by substituting Example 5A and 4-{[5-(chloromethyl)-1H-imidazol-1-yl]methyl}benzonitrile (prepared according to the procedure described in WO 00/01691) for Example 1G and Example 1I, respectively, in Example 1J. MS (DCI) m/z 480 (M+H)⁺; ¹H NMR (300 MHz, CDCl₃) δ7.60 (s, 1H), 7.57 (d, J=9 Hz, 2H), 7.07 (d, J=9 Hz, 2H), 6.98 (s, 1H), 6.82-6.86 (m, 1H), 6.65-6.70 (m, 2H), 6.40 (s, 1H), 6.04 (s, 2H), 5.27 (s, 2H), 3.52 (s, 3H), 3.30 (s, 2H), 3.10 (s, 2H), 2.64 (s, 4H); Anal. Calcd for C₂₈H₂₅N₅O₃: C, 70.13; H, 5.25; N, 14.60. Found: C, 69.82; H, 5.06; N, 14.27.

### EXAMPLE 7

4-[(5-{[3-(3-chlorophenyl)-1-methyl-1,4,6,7-tetrahydro-5H-pyrazolo[4,3-c]pyridin-5-yl]methyl}-1H-imidazol-1-yl)methyl]benzonitrile

### EXAMPLE 7A

1-acetyl-3-(3-chlorobenzoyl)piperidin-4-one

The desired product was prepared according to the procedure described in Eur. J. Med. Chem. 1992, 27, 655-661.

### EXAMPLE 7B

5-acetyl-3-(3-chlorophenyl)-4,5,6,7-tetrahydro-1H-pyrazolo [43-c]pyridine

The desired product was prepared according to the procedure described in J. Med. Chem. 1985, 28, 934-940.

### EXAMPLE 7C

5-acetyl-3-(3-chlorophenyl)-1-methyl-4,5,6,7-tetrahydro-1H-pyrazolo[4,3-c]pyridine

A solution of Example 7B (200 mg, 0.73 mmol) in DMF (5 mL) at room temperature was treated with 60% NaH dispersion (45 mg, 1.09 mmol), stirred for 15 minutes, treated with CH₃I (100 μL, 228 mg, 1.61 mmol), stirred overnight, diluted with water, and extracted three times with ethyl acetate. The combined extracts were washed with water and brine, dried (MgSO₄), filtered, and concentrated. The concentrate was purified by flash column chromatography on silica gel with 2% methanol/dichloromethane to provide 110 mg (52%) of the desired product.

### EXAMPLE 7D

3-(3-chlorophenyl)-1-methyl-4,5,6,7-tetrahydro-1H-pyrazolo[4,3-c]pyridine

A suspension of Example 7C (100 mg, 0.35 mmol) in 10% HCl (aq) (8 mL) was heated to reflux overnight, neutralized with saturated NaHCO₃ (aq), and extracted three times with ethyl acetate. The combined extracts were dried (MgSO₄), filtered, and concentrated to provide 75 mg (88%) of the desired product.

### EXAMPLE 7E

4-[(5-{[3-(3-chlorophenyl)-1-methyl-1,4,6,7-tetrahydro-5H-pyrazolo[4,3-c]pyridin-5-yl]methyl}-1H-imidazol-1-yl)methyl]benzonitrile

The desired product was prepared as the free base by substituing Example 7D and 4-{[5-(chloromethyl)-1H-imidazol-1-yl]methyl}benzonitrile (prepared according to the procedure described in WO 00/01691) for Example 1G and Example 1I, respectively, in Example 1J. MS (DCI) m/z 443 (M+H)⁺; ¹H NMR (300 MHz, DMSO-d₆) δ7.77 (s, 1H), 7.60 (d, J=9 Hz, 2H), 7.53-7.56 (m, 1H), 7.40-7.44 (m, 2H), 7.30-7.36 (m, 1H), 7.18 (d, J=9 Hz, 2H), 6.91 (s, 1H), 5.33 (s, 2H), 3.70 (s, 3H), 3.58 (s, 2H), 3.44 (s, 2H), 2.46-2.63 (m, 4H); Anal. Calcd for C₂₅H₂₃ClN₆.0.5H₂O: C, 66.44; H, 5.35; N, 18.59. Found: C, 66.19; H, 5.15; N, 18.33.

It will be evident to one skilled in the art that the present invention is not limited to the foregoing illustrative examples, and that it can be embodied in other specific forms without departing from the essential attributes thereof. It is therefore desired that the examples be considered in all respects as illustrative and not restrictive, reference being made to the appended claims, rather than to the foregoing examples, and all changes which come within the meaning and range of equivalency of the claims are therefore intended to be embraced therein.

What is claimed is

1. A compound of formula (I) 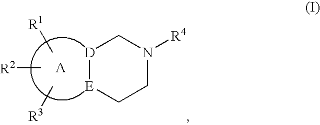
or a therapeutically acceptable salt thereof, wherein
A is a 5- or 6-membered aromatic or non-aromatic ring wherein from 0-3 carbon atoms are replaced by nitrogen;
D and E are independently selected from the group consisting of C and N;
with the proviso that when one of D and E is N, the other is C;
R¹ is selected from the group consisting of aryl and heteroaryl;
R² and R³ are selected from the group consisting of hydrogen, alkenyl, alkoxy, alkyl, amido, amino, cyano, halo, haloalkoxy, haloalkyl, nitro, and oxo; and
R⁴ is selected from the group consisting of arylalkyl and heteroarylalkyl.

2. The compound according to claim 1 of formula (II) 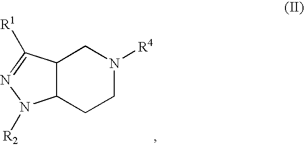
or a therapeutically acceptable salt thereof, wherein
R¹ is selected from the group consisting of aryl and heteroaryl;
R² is selected from the group consisting of hydrogen, alkenyl, and alkyl; and
R⁴ is selected from the group consisting of arylalkyl and heteroarylalkyl.

3. The compound according to claim 2 wherein
R¹ is aryl;
R² is alkyl; and
R⁴ is heteroarylalkyl.

4. The compound according to claim 3 which is
4-[(5-{[3-(3-chlorophenyl)-1-methyl-1,4,6,7-tetrahydro-5H-pyrazolo[4,3-c]pyridin-5-yl]methyl}-1H-imidazol-1-yl)methyl]benzonitrile.

5. The compound according to claim 1 of formula (III) 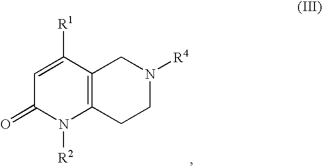
or a therapeutically acceptable salt thereof, wherein
R¹ is selected from the group consisting of aryl and heteroaryl;
R² is selected from the group consisting of hydrogen, alkenyl, and alkyl; and
R⁴ is selected from the group consisting of arylalkyl and heteroarylalkyl.

6. The compound according to claim 5 wherein
R¹ is aryl;
R² is alkyl; and
R⁴ is heteroarylalkyl.

7. The compound according to claim 6 selected from the group consisting of 4-[[4-(3-chlorophenyl)-1-methyl-2-oxo-1,5,7,8-tetrahydro-1,6-naphthyridin-6(2H)-yl](1-methyl-1H-imidazol-5-yl)methyl]benzonitrile; 4-[(5-{[4-(3-chlorophenyl)-1-methyl-2-oxo-1,5,7,8-tetrahydro-1,6-naphthyridin-6(2H)-yl]methyl}-1H-imidazol-1-yl)methyl]benzonitrile; 4-[[4-(3-methoxyphenyl)-1-methyl-2-oxo-1,5,7,8-tetrahydro-1,6-naphthyridin-6(2H)-yl](1-methyl-1H-imidazol-5-yl)methyl]benzonitrile; 4-[(5-{[4-(3-methoxyphenyl)-1-methyl-2-oxo-1,5,7,8-tetrahydro-1,6-naphthyridin-6(2H)-yl]methyl}-1H-imidazol-1-yl)methyl]benzonitrile; 4-[[4-(1,3-benzodioxol-5-yl)-1-methyl-2-oxo-1,5,7,8-tetrahydro-1,6-naphthyridin-6(2H)-yl](1-methyl-1H-imidazol-5-yl)methyl]benzonitrile; and 4-[(5-{[4-(1,3-benzodioxol-5-yl)-1-methyl-2-oxo-1,5,7,8-tetrahydro-1,6-naphthyridin-6(2H)-yl]methyl}-1H-imidazol-1-yl)methyl]benzonitrile.

8. A pharmaceutical composition comprising a compound of claim 1 or a therapeutically acceptable salt thereof, in combination with a therapeutically acceptable carrier.

9. A method for inhibiting farnesyltransferase in a patient in recognized need of such treatment comprising administering to the patient a therapeutically acceptable amount of a compound of claim 1, or a therapeutically acceptable salt thereof.

10. A method for treating cancer in a patient in recognized need of such treatment comprising administering to the patient a therapeutically acceptable amount of a compound of claim 1, or a therapeutically acceptable salt thereof.
